(19) United States
(12) Patent Application Publication
(10) Pub. No.: US 2018/0371163 A1
(43) Pub. Date: Dec. 27, 2018

(54) METHOD FOR PRODUCING OLIGOMERS FOR PRODUCING POLYCARBONATES

(71) Applicant: SHELL OIL COMPANY, HOUSTON, TX (US)

(72) Inventors: Garo Garbis VAPORCIYAN, Houston, TX (US); Kunquan YU, Katy, TX (US)

(21) Appl. No.: 16/064,401

(22) PCT Filed: Dec. 20, 2016
(86) PCT No.: PCT/US2016/067659

§ 371 Date: Jun. 20, 2018

Related U.S. Application Data
(60) Provisional application No. 62/270,719, filed on Dec. 22, 2015.

(51) Int. Cl. C08G 64/30; C08K 5/07; C07C 68/06; B01J 27/232; B01J 23/04

(52) U.S. Cl. CPC C08G 64/305; C08K 5/07; C08G 64/307; C08G 2105/00; B01J 27/232; B01J 23/04; B01J 2231/49; C07C 68/06

(57) ABSTRACT

A process for producing an oligomer comprising contacting a alkylaryl carbonate and a dihydroxy compound in a reaction zone in the presence of an oligomerization catalyst under oligomerization conditions to form the oligomer wherein the molar ratio of dihydroxy compound to alkylaryl carbonate in the reaction zone is at least 2:1.

### REFERENCE TO RELATED APPLICATION

The present application claims the benefit of U.S. Provisional Patent Application Ser. No. 62/270,719, filed Dec. 22, 2015.

### FIELD OF THE INVENTION

This invention relates to a method of producing an oligomer from an alkylaryl carbonate and a dihydroxy compound.

### BACKGROUND OF THE INVENTION

Aromatic polycarbonate, further referred to herein as polycarbonate, is a widely used raw material in many different manufacturing sectors. Due to the hardness and transparency of the material, it can be applied in applications as diverse as automotive windows and optical lenses. It is believed that the demand for polycarbonate will increase significantly in the coming years, requiring improvements in the production of polycarbonate, particularly in terms of efficiency and environmental impact.

Several processes for the production of polycarbonate are known. For instance, a process including reacting phosgene and 2,2-bis(4-hydroxyphenyl)propane (BPA) under phase transfer conditions is applied on an industrial scale. However, this process has the inherent drawbacks of employing the toxic component phosgene and creating chloride containing waste streams.

A different process that does not require the use of phosgene is based on the transesterification of BPA with dialkyl carbonate or diaryl carbonate. The use of a dialkyl carbonate has the disadvantage that in the transesterification with bisphenolacetone, it is not reactive enough under commercially reasonable conditions, to form sufficient quantities of polymeric polycarbonate. Furthermore, the alkyl alcohol that is liberated is not used in any other part of the process for producing polycarbonate, and recycling of the alkyl alcohol to the dialkyl carbonate production requires substantial purification.

The use of a diaryl carbonate, in particular diphenyl carbonate (DPC), has the advantage that it is reactive enough to form polymeric polycarbonate. Furthermore, phenol is liberated in the reaction of the diphenyl carbonate with bisphenolacetone to form polycarbonate, for instance as described in U.S. Pat. No. 5,589,564. This phenol may in turn be recycled to the production of bisphenolacetone or diphenyl carbonate, for which it is a main raw material. Diphenyl carbonate is expensive and it is desirable to find a way to carry out this process without the substantial cost of using large amounts of diphenyl carbonate. The above process for production of polycarbonate leaves ample room for improvement, in particular in view of the raw materials that are used.

JP S64-16826 describes a process for producing polycarbonate comprising three steps. In the first step, bisphenol acetone is reacted with a dialkyl carbonate at a ratio in the range of 1:1 to 1:100. This reaction produces a dialkyl biscarbonate of bisphenol acetone which is then reacted with an equimolar or greater amount of diphenyl carbonate to produce polycarbonate. In the third step, alkyl phenyl carbonate produced as a byproduct is converted to diphenyl carbonate and dialkyl carbonate.

### SUMMARY OF THE INVENTION

This invention provides a process for producing an oligomer comprising contacting an alkylaryl carbonate and a dihydroxy compound in a reaction zone in the presence of an oligomerization catalyst under oligomerization conditions to form the oligomer wherein the molar ratio of dihydroxy compound to alkylaryl carbonate in the reaction zone is at least 2:1.

### DETAILED DESCRIPTION OF THE INVENTION

The invention provides a new way to form oligomers that can be used to form polycarbonates. The process comprises contacting an excess of a dihydroxy compound with an alkylaryl carbonate to produce an oligomer that can be used in a further process to produce polycarbonate. The oligomer is preferably a dihydroxy capped carbonate, for example a carbonate with a BPA molecule on each end. In this application, the oligomer may be a monomer or more than one monomer linked together.

The dihydroxy compound that is used in the process can be an aliphatic diol, an acid or a dihydroxy aromatic compound.

The dihydroxy compound may comprise one or more aliphatic diols. Embodiments of suitable aliphatic diols include: isosorbide; 1,4:3,6-dianhydro-D-sorbitol; tricyclodecane-dimethanol; 4,8-bis(hydroxymethyl) tricyclodecane; tetramethylcyclobutanediol; 2,2,4,4-tetramethylcyclobutane-1,3-diol; cis/trans-1,4-cyclohexanedimethanol; cyclohex-1,4-ylenedimethanol; trans-1,4-cyclohexanedimethanol; trans-1,4-bis(hydroxymethyl) cyclohexane; cis-1,4-cyclohexanedimethanol; cis-1,4-bis(hydroxymethyl) cyclohexane; cis-1,2-cyclohexanedimethanol; 1,1′-bi(cyclohexyl)-4,4′-diol; dicyclohexyl-4,4′-diol; 4,4′-di-hydroxybicyclohexyl; and poly(ethylene glycol).

The dihydroxy compound may comprise one or more acids. Embodiments of suitable acids include: 1,10-dodecanoic acid; adipic acid; hexanedioic acid, isophthalic acid; 1,3-benzenedicarboxylic acid; teraphthalic acid; 1,4-benzenedicarboxylic acid; 2,6-naphthalenedicarboxylic acid; 3-hydroxybenzoic acid; and 4-hydroxybenzoic acid.

The dihydroxy compound may comprise one or more dihydroxy aromatic compounds. A dihydroxy aromatic compound is an aromatic compound comprising two hydroxyl groups on one or more aromatic rings. Examples of dihydroxy aromatic compounds include bisphenol, for example, BPA, which is a preferred dihydroxy aromatic compound and dihydroxy benzene, for example resorcinol.

Dihydroxy aromatic compounds can be bisphenols having one or more halogen, nitro, cyano, alkyl, or cycloalkyl groups. Embodiments of suitable bisphenols include 2,2-bis(4-hydroxyphenyl) propane (BPA); 2,2-bis(3-chloro-4-hydroxyphenyl) propane; 2,2-bis(3-bromo-4-hydroxyphenyl) propane; 2,2-bis(4-hydroxy-3-methylphenyl) propane; 2,2-bis (4-hydroxy-3-isopropylphenyl) propane; 2,2-bis(3-t-butyl-4-hydroxyphenyl) propane; 2,2-bis(3-phenyl-4-hydroxyphenyl) propane; 2,2-bis(3,5-dichloro-4-hydroxyphenyl) propane; 2,2-bis(3,5-dibromo-4-hydroxyphenyl) propane; 2,2-bis(3,5-dimethyl-4-hydroxyphenyl) propane; 2,2-bis(3-chloro-4-hydroxy-5-methylphenyl) propane; 2,2-bis(3-bromo-4-hydroxy-5-methylphenyl) propane; 2,2-bis(3-chloro-4-hydroxy-5-isopropylphenyl) propane; 2,2-bis(3-bromo-4-hydroxy-5-isopropylphenyl) propane; 2,2-bis(3-t-butyl-5-chloro-4-hydroxyphenyl) propane; 2,2-bis(3-bromo-5-t-butyl-4-hydroxyphenyl) propane; 2,2-bis(3-chloro-5-phenyl-4-hydroxyphenyl) propane; 2,2-bis(3-bromo-5-phenyl-4-hydroxyphenyl) propane; 2,2-bis(3,5-di-isopropyl-1-4-hydroxyphenyl) propane; 2,2-bis(3,5-di-t-butyl-4-hydroxyphenyl) propane; 2,2-bis(3,5-diphenyl-4-hydroxyphenyl) propane; 2,2-bis(4-hydroxy-2,3,5,6-tetrachlorophenyl) propane; 2,2-bis(4-hydroxy-2,3,5,6-tetrabromophenyl) propane; 2,2-bis(4-hydroxy-2,3,5,6-tetramethylphenyl) propane; 2,2-bis(2,6-dichloro-3,5-dimethyl-4-hydroxyphenyl) propane; 2,2-bis(2,6-dibromo-3,5-dimethyl-4-hydroxyphenyl) propane; 1,1-bis(4-hydroxyphenyl) cyclohexane; 1,1-bis(3-chloro-4-hydroxyphenyl) cyclohexane; 1,1-bis(3-bromo-4-hydroxyphenyl) cyclohexane; 1,1-bis(4-hydroxy-3-methylphenyl) cyclohexane; 1,1-bis(4-hydroxy-3-isopropylphenyl) cyclohexane; 1,1-bis(3-t-butyl-4-hydroxyphenyl) cyclohexane; 1,1-bis (3-phenyl-4-hydroxyphenyl) cyclohexane; 1,1-bis(3,5-dichloro-4-hydroxyphenyl) cyclohexane; 1,1-bis (3,5-dibromo-4-hydroxyphenyl) cyclohexane; 1,1-bis(3,5-dimethyl-4-hydroxyphenyl) cyclohexane; 1,1-bis (3-chloro-4-hydroxy-5-methylphenyl) cyclohexane; 1,1-bis(3-bromo-4-hydroxy-5-methylphenyl) cyclohexane; 1,1-bis(3-chloro-4-hydroxy-5-isopropylphenyl) cyclohexane; 1,1-bis(3-bromo-4-hydroxy-5-isopropylphenyl) cyclohexane; 1,1-bis(3-t-butyl-5-chloro-4-hydroxyphenyl) cyclohexane; 1,1-bis (3-bromo-5-t-butyl-4-hydroxyphenyl) cyclohexane; 1,1-bis (3-chloro-5-phenyl-4-hydroxyphenyl) cyclohexane; 1,1-bis(3-bromo-5-phenyl-4-hydroxyphenyl) cyclohexane; 1,1-bis(3,5-di sopropyl-4-hydroxyphenyl) cyclohexane; 1,1-bis(3,5-di-t-butyl-4-hydroxyphenyl) cyclohexane; 1,1-bis(3,5-diphenyl-4-hydroxyphenyl) cyclohexane; 1,1-bis(4-hydroxy-2,3,5,6-tetrachlorophenyl) cyclohexane; 1,1-bis(4-hydroxy-2,3,5,6-tetrabromophenyl) cyclohexane; 1,1-bis(4-hydroxy-2,3,5,6-tetramethylphenyl) cyclohexane; 1,1-bis(2,6-dichloro-3,5-dimethyl-4-hydroxyphenyl) cyclohexane; 1,1-bis(2,6-dibromo-3,5-dimethyl-4-hydroxyphenyl) cyclohexane; 1,1-bis(4-hydroxyphenyl)-3,3,5-trimethylcyclohexane; 1,1-bis (3-chloro-4-hydroxyphenyl)-3,3,5-trimethylcyclohexane; 1,1-bis(3-bromo-4-hydroxyphenyl)-3,3,5-trimethylcyclohexane; 1,1-bis(4-hydroxy-3-methylphenyl)-3,3,5-trimethylcyclohexane; 1,1-bis(4-hydroxy-3-isopropylphenyl)-3,3,5-trimethylcyclohexane; 1,1-bis(3-t-butyl-4-hydroxyphenyl)-3,3,5-trimethylcyclohexane; 1,1-bis (3-phenyl-4-hydroxyphenyl)-3,3,5-trimethylcyclohexane; 1,1-bis(3,5-dichloro-4-hydroxyphenyl)-3,3,5-trimethylcyclohexane; 1,1-bis(3,5-dibromo-4-hydroxyphenyl)-3,3,5-trimethylcyclohexane; 1,1-bis (3,5-dimethyl-4-hydroxyphenyl)-3,3,5-trimethylcyclohexane; 1,1-bis (3-chloro-4-hydroxy-5-methylphenyl)-3,3,5-trimethylcyclohexane; 1,1-bis(3-bromo-4-hydroxy-5-methylphenyl)-3,3,5-trimethylcyclohexane; 1,1-bis (3-chloro-4-hydroxy-5-isopropylphenyl)-3,3,5-trimethylcyclohexane; 1,1-bis(3-bromo-4-hydroxy-5-isopropylphenyl)-3,3,5-trimethylcyclohexane; 1,1-bis(3-t-butyl-5-chloro-4-hydroxyphenyl)-3,3,5-trimethylcyclohexane; 1,1-bis(3-bromo-5-t-butyl-4-hydroxyphenyl)-3,3,5-trimethylcyclohexane; bis(3-chloro-5-phenyl-4-hydroxyphenyl)-3,3,5-trimethylcyclohexane; 1,1-bis(3-bromo-5-phenyl-4-hydroxyphenyl)-3,3,5-trimethylcyclohexane; 1,1-bis(3,5-di-isopropyl-4-hydroxyphenyl)-3,3,5-trimethylcyclohexane; 1,1-bis(3,5-di-t-butyl-4-hydroxyphenyl)-3,3,5-trimethylcyclohexane; 1,1-bis(3,5-diphenyl-4-hydroxyphenyl)-3,3,5-trimethylcyclohexane; 1,1-bis(4-hydroxy-2,3,5,6-tetrachlorophenyl)-3,3,5-trimethylcyclohexane; 1,1-bis(4-hydroxy-2,3,5,6-tetrabromophenyl)-3,3,5-trimethylcyclohexane; 1,1-bis(4-hydroxy-2,3,5,6-tetramethylphenyl)-3,3,5-trimethylcyclohexane; 1,1-bis(2,6-dichloro-3,5-dimethyl-4-hydroxyphenyl)-3,3,5-trimethylcyclohexane; 1,1-bis(2,6-dibromo-3,5-dimethyl-4-hydroxyphenyl)-3,3,5-trimethylcyclohexane; 4,4′-dihydroxy-1,1-biphenyl; 4,4′-dihydroxy-3,3′-dimethyl-1,1-biphenyl; 4,4′-dihydroxy-3,3′-dioctyl-1,1-biphenyl; 4,4′-dihydroxydiphenylether; 4,4′-dihydroxydiphenylthioether; 1,3-bis (2-(4-hydroxyphenyl)-2-propyl) benzene; 1,3-bis(2-(4-hydroxy-3-methylphenyl)-2-propyl) benzene; 1,4-bis(2-(4-hydroxyphenyl)-2-propyl) benzene and 1,4-bis(2-(4-hydroxy-3-methylphenyl)-2-propyl) benzene.

Embodiments of suitable dihydroxy benzenes include hydro-quinone, resorcinol, methylhydroquinone, butylhydro-quinone, phenylhydroquinone, 4-phenylresorcinol and 4-methylresorcinol.

Embodiments of suitable dihydroxy naphthalenes include 2,6-dihydroxy naphthalene; 2,6-dihydroxy-3-methyl naphthalene; 2,6-dihydroxy-3-phenyl naphthalene; 1,4-dihydroxy naphthalene; 1,4-dihydroxy-2-methyl naphthalene; 1,4-dihydroxy-2-phenyl naphthalene and 1,3-dihydroxy naphthalene.

The alkylaryl carbonate is represented by the formula R¹OCOOR². R¹ represents an alkyl group having 1 to 10 carbon atoms, an alicyclic group having 3 to 10 carbon atoms or an aralkyl group having 6 to 10 carbon atoms. R² represents an alkyl group having 1 to 15 carbon atoms.

Examples of R¹ include an alkyl group, such as methyl, ethyl, propyl, allyl, butyl, butenyl, pentyl, hexyl, heptyl, octyl, nonyl, decyl and cyclohexylmethyl and isomers thereof. Further examples of R¹ include an alicyclic group, such as cyclopropyl, cyclobutyl, cyclopentyl, cyclohexyl, and cycloheptyl; and an aralkyl group, such as benzyl, phenethyl, phenylpropyl, phenylbutyl, methylbenzyl and isomers thereof. The alkyl, alicyclic or aralkyl group may be substituted with a substituent such as a lower alkyl group, a lower alkoxy group, a cyano group and a halogen atom.

Examples of R² include an aryl group, such as benzyl, tolyl, xylyl as well as substituted aryl groups. The aryl group may be substituted with nitrogen, sulfur, halides or compounds thereof.

Examples of the alkylaryl carbonate are methylphenyl carbonate, methyltolyl carbonate, ethylphenyl carbonate, and butylphenyl carbonate.

An alkylaryl carbonate where R¹ is an alkyl group having four or less carbon atoms is preferred. The alkylaryl carbonate is most preferably ethylphenyl carbonate.

The alkylaryl carbonate may be produced by any method known to one of ordinary skill in the art. For example, the alkylaryl carbonate may be produced by contacting an alkylene carbonate and a phenol feedstock in a reaction zone to react in the presence of a transesterification catalyst to yield an alkanediol-rich stream and a stream comprising alkylaryl carbonate and alkanol which streams are separated by one or more steps to produce an alkylaryl carbonate rich stream.

The oligomerization catalyst used in the reaction of these reactants can be any known transesterification catalyst. The catalyst can be heterogeneous or homogeneous. In another embodiment, both heterogeneous and homogeneous catalysts may be used.

The catalyst may include hydrides, oxides, hydroxides, alcoholates, amides or salts of alkali metals, i.e., lithium, sodium, potassium, rubidium and cesium. The catalyst may be a hydroxide or alcoholate of potassium or sodium. Other suitable catalysts are alkali metal salts, for example, acetates, propionates, butyrates or carbonates.

Further suitable catalysts include phosphines, arsines or divalent sulfur compounds and selenium compounds and onium salts thereof. Examples of this type of catalyst includes tributylphosphine; triphenylphosphine; diphenylphosphine; 1,3-bis(diphenylphosphino) propane; triphenylarsine; trimethylarsine; tributylarsine; 1,2-bis(diphenylarsino) ethane; triphenylantimony; diphenylsulfide; diphenyldisulfide; diphenylselenide; tetraphenylphosphonium halide (Cl, Br, I); tetraphenylarsonium halide (Cl, Br, I); triphenylsulphonium halide (Cl, Br, I).

Additional suitable catalysts include complexes or salts of tin, titanium or zirconium. Examples of this type of catalyst include butylstannonic acid; tin methoxide; dimethyltin; dibutyltin oxide; dibutyltin dilaurate; tributyltin hydride; tributyltin chloride; tin(II) ethylhexanoates; zirconium alkoxides (methyl, ethyl or butyl); zirconium(IV) halides (F, Cl, Br, I); zirconium nitrates; zirconium acetylacetonate; titanium alkoxides (methyl, ethyl or isopropyl); titanium acetate; titanium acetylacetonate.

The catalyst may be an ion exchange resin that contains suitable functional groups, for example, tertiary amine groups, quaternary ammonium groups, sulfonic acid groups and carboxylic acid groups. The catalyst may be an alkali metal or alkaline earth metal silicate. The catalyst may comprise an element from Group 4 (such as titanium), Group 5 (such as vanadium), Group 6 (such as chromium or molybdenum) or Group 12 (such as zinc) of the Periodic Table of the Elements, or tin or lead, or a combination of such elements, such as a combination of zinc with chromium (for example zinc chromite). These elements may be present in the catalyst as an oxide, such as zinc oxide.

The catalyst may be selected from the group consisting of sodium hydroxides, sodium carbonates, lithium hydroxides, lithium carbonates, tetraalkylammonium hydroxides, tetraalkylammonium carbonates, titanium alkoxides, lead alkoxides, tin alkoxides and aluminophosphates.

The contacting of the dihydroxy compound and the alkylaryl carbonate can take place in a batch, semi-batch or continuous reaction step. The oligomerization reaction may be carried out in any type of reactor, for example, a batch reactor, a batch reactor with a vacuum withdrawal, a batch reactor with a distillation column; or a catalytic distillation column. The reaction is preferably carried out in a reactor that provides for the removal of alcohol during the reaction. The reaction is an equilibrium reaction, and the removal of alcohol shifts the equilibrium in favor of the desired products.

In a catalytic or reactive distillation column, the reaction takes place in the same place that the separation of reactants and products takes place. In this column, there is a reaction zone that can be defined as the portion of the reactive distillation column where catalyst is present. This catalyst may be homogeneous or heterogeneous.

The reaction can be carried out in multiple batch reactors that are operated with their operating cycles out of synchronization. In this way, product would be produced continuously and any further reaction steps could be carried out continuously.

In an embodiment of a semi-batch operation, the dihydroxy compound, the alkylaryl carbonate and the catalyst can be combined in a stirred pot reactor. The reactor can be connected to a distillation apparatus that removes alcohol that is formed as part of the reaction. This shifts the equilibrium towards the products and improves the performance of the reaction. If alkylaryl carbonate is removed via the distillation apparatus, it can be recycled to the reactor.

The first addition product formed by the reaction is an alkyl-dihydroxy-carbonate intermediate or an aryl-dihydroxy-carbonate intermediate. For example, if the dihydroxy compound is BPA and the alkylaryl carbonate is methylphenyl carbonate, then the intermediate formed would be methyl-BPA-carbonate or phenyl-BPA-carbonate.

The intermediate is further reacted, either via disproportionation or via further transesterification with an additional dihydroxy compound. The disproportionation reaction would result in producing dialkyl carbonate, alkylaryl carbonate or diaryl carbonate. The further transesterification would result in production of a carbonate molecule capped on both ends with a dihydroxy compound.

The overall reaction is conducted with an excess of dihydroxy compound to ensure that there is sufficient dihydroxy compound to produce the dihydroxy capped carbonate. For example, if the dihydroxy compound is BPA and the alkylaryl carbonate is ethylphenyl carbonate, the reaction will produce BPA capped carbonate.

The reaction is carried out to produce as much of the dihydroxy capped carbonate as possible. The first intermediate, alkyl-dihydroxy-carbonate or aryl-dihydroxy-carbonate is produced, but the reaction is conducted to minimize the amount of alkyl-dihydroxy-carbonate or aryl-dihydroxy-carbonate remaining at the end of the reaction.

The oligomerization conditions of the reaction step can be adjusted to provide for removal of the alcohol formed and also to ensure adequate reaction rates. If the temperature is too high or the pressure too low, then the reactants may be carried out of the reaction zone via the distillation apparatus or side reactions may be promoted.

The oligomerization is preferably carried out at a pressure of less than 2.03 MPa. The pressure is preferably in a range of from 101.3 kPa to 2.03 MPa. The oligomerization is preferably carried out at a temperature in the range of from 110° C. to 330° C., preferably of from 160° C. to 300° C., and most preferably of from 180° C. to 280° C.

Reactor conditions may be changed as the reaction proceeds. Initially, the temperature and pressure need to be such that the temperature is high enough to drive the reaction and evaporate any alcohol formed. The temperature should not be too high as it will also evaporate the alkylaryl carbonate before it reacts with the dihydroxy compound. In addition, higher temperatures can result in undesired side reactions.

It is preferred to use an excess of the dihydroxy compound to ensure that the reaction proceeds to produce the dihydroxy capped carbonate. The feed to the reactor comprises a dihydroxy compound and alkylaryl carbonate at a molar ratio of at least 2:1. The dihydroxy compound to alkylaryl carbonate molar ratio is preferably at least 3:1, more preferably 5:1 and most preferably 10:1. The dihydroxy compound to alkylaryl carbonate molar ratio is preferably in a range of from 2:1 to 100:1, preferably in a range of from 5:1 to 50:1.

Due to the excess of dihydroxy compound used, it is preferred to remove some or all of the excess dihydroxy compound after the reaction is conducted and the dihydroxy capped carbonate is formed. This provides for a purer dihydroxy capped carbonate product that can be used in further reaction steps if desired. In another embodiment, the excess dihydroxy compound can be left with the dihydroxy capped carbonate.

### Products and Byproducts

Alcohol may be formed during the reaction. For example, if ethylphenyl carbonate is used as the alkylaryl carbonate, then ethanol and/or phenol will be formed. In addition, other byproducts may be formed, including isomers of the oligomer.

The oligomer formed in this reaction may be further reacted with the same or a different alkylaryl carbonate.

1. A process for producing an oligomer comprising contacting an alkylaryl carbonate and a dihydroxy compound in a reaction zone in the presence of an oligomerization catalyst under oligomerization conditions to form the oligomer wherein the molar ratio of dihydroxy compound to alkylaryl carbonate in the reaction zone is at least 2:1.

2. The process of claim 1, wherein the alkylaryl carbonate is selected from the group consisting of methylphenyl carbonate, ethylphenyl carbonate and mixtures thereof.

3. The process of claim 1, wherein the dihydroxy compound is selected from the group consisting of aliphatic diols, acids and dihydroxy aromatics.

4. The process of claim 1, wherein the dihydroxy compound is selected from the group consisting of bisphenols, dihydroxy benzenes and dihydroxy naphthalenes.

5. The process of claim 1, wherein the ratio of dihydroxy compound to alkylaryl carbonate in the reaction zone is at least 5:1.

6. The process of claim 1, wherein the ratio of dihydroxy compound to alkylaryl carbonate in the reaction zone is at least 10:1.

7. The process of claim 1, wherein the ratio of dihydroxy compound to alkylaryl carbonate in the reaction zone is in the range of from 2:1 to 100:1.

8. The process of claim 1, further comprising removing at least a portion of unreacted dihydroxy compound from the oligomer.

9. The process of claim 1, wherein an alcohol is formed during the oligomerization.

10. The process of claim 9, wherein the oligomerization conditions comprise a temperature and pressure at which at least a portion of the alcohol is in the vapor phase.

11. The process of claim 1, wherein the oligomerization conditions comprise a pressure of less than 2.03 MPa.

12. The process of claim 1, wherein the oligomerization conditions comprise a temperature in the range of from 110 to 330° C.

13. The process of claim 1, wherein the oligomerization conditions comprise a temperature in the range of from 160 to 300° C.

14. The process of claim 1, wherein the oligomerization is carried out in a plurality of reactors.

15. The process of claim 1, wherein the oligomerization reaction is carried out as a batch process.

16. The process of claim 1, further comprising contacting the oligomer with additional alkylaryl carbonate in a separate reaction zone.

17. The process of claim 1, wherein the oligomerization catalyst is heterogeneous.

18. The process of claim 1, wherein the oligomerization catalyst is homogeneous.

19. The process of claim 1, wherein the oligomerization catalyst is selected from the group consisting of sodium hydroxides, sodium carbonates, lithium hydroxide, lithium carbonates, tetraalkylammonium hydroxides, tetraalkylammonium carbonates and titanium alkoxides.
